(19) United States
(12) Patent Application Publication
(10) Pub. No.: US 2011/0200171 A1
(43) Pub. Date: Aug. 18, 2011

(54) METHODS AND APPARATUS FOR RENAL NEUROMODULATION VIA STEREOTACTIC RADIOTHERAPY

(75) Inventors: Robert J. Beetel, Redwood City, CA (US); Neil C. Barman, Menlo Park, CA (US); Benjamin J. Clark, Redwood City, CA (US); Paul Friedrichs, Belmont, CA (US); Kenneth J. Michlitsch, Livermore, CA (US); Karun D. Naga, Santa Clara, CA (US); Andrew Wu, Foster City, CA (US); Denise Zarins, Saratoga, CA (US)

(73) Assignee: Ardian, Inc., Mountain View, CA (US)

(21) Appl. No.: 13/009,748

(22) Filed: Jan. 19, 2011

Related U.S. Application Data
(60) Provisional application No. 61/296,417, filed on Jan. 19, 2010.

(51) Int. Cl. A61N 5/10

(52) U.S. Cl. 378/65

(57) ABSTRACT

The present disclosure describes methods and apparatus for renal neuromodulation via stereotactic radiotherapy for the treatment of hypertension, heart failure, chronic kidney disease, diabetes, insulin resistance, metabolic disorder or other ailments. Renal neuromodulation may be achieved by locating renal nerves and then utilizing stereotactic radiotherapy to expose the renal nerves to a radiation dose sufficient to reduce neural activity. A neural location element may be provided for locating target renal nerves, and a stereotactic radiotherapy system may be provided for exposing the located renal nerves to a radiation dose sufficient to reduce the neural activity, with reduced or minimized radiation exposure in adjacent tissue. Renal nerves may be located and targeted at the level of the ganglion and/or at postganglionic positions, as well as at pre-ganglionic positions.

### REFERENCE TO RELATED APPLICATIONS

The present application claims the benefit of U.S. Provisional Patent Application No. 61/296,417, filed on Jan. 19, 2010, the disclosure of which is incorporated herein by reference in its entirety.

### TECHNICAL FIELD

The technology disclosed in the present application generally relates to methods and apparatus for renal neuromodulation via stereotactic radiotherapy.

### BACKGROUND

Radiation therapy, or radiotherapy, comprising directed external beams of radiation, has been used for some time in the treatment of cancer and various other ailments to non-invasively destroy malignant tissue. Radiotherapy may be delivered to target tissue during a single procedure in a single fraction (often referred to as radiosurgery), or may be delivered during multiple procedures using a multi-fraction approach. Radiation beams may be derived from active radiation sources, such as alpha, beta or gamma radiation sources, or may be actively generated using a particle accelerator, such as a linear accelerator (“LINAC”). LINAC-derived irradiation may comprise an accelerated electron beam for treatment of superficial or surgically exposed ailments, or may comprise high energy X-rays for penetrating through tissue to target deeper seated ailments.

In order to increase the radiation dose delivered to target tissue, while concurrently reducing the dose delivered to adjacent normal tissue, conformal radiotherapy and intensity-modulated radiotherapy (IMRT) techniques have been developed. Such techniques clearly define the 3-dimensional structure and location of target tissue and then precisely deliver radiation to that 3-dimensional tissue volume with increased intensity, as compared to the intensity delivered to surrounding normal tissue. Stereotactic radiotherapy accomplishes such preferential radiation delivery by utilizing a plethora of relatively low dose radiation pulses that are delivered to the target tissue from a variety of directions. The pulses can be delivered in complex, overlapping patterns that conform to irregularly shaped tumor volumes. The relatively low dose irradiation delivered from various directions by these pulses accumulates in the targeted tissue volume to provide a desired higher radiation dose that is sufficient to destroy all or part of the malignant tissue. Beneficially, a steep fall-off gradient of the target dose yields significantly lower radiation exposure in adjacent normal tissue.

Stereotactic radiotherapy is used in the treatment of brain tumors, for example, using the Gamma Knife® (Elekta AB; Stockholm, Sweden). The patient is immobilized to reduce or mitigate migration of the tumor relative to a fixed isocenter coordinate system. Radiopaque boney landmarks and/or external frames are used as reference points, which may be combined with pre-treatment MRI and/or CT data to locate the position of the tumor in free space and direct the low dose, multi-directional pulses delivered from the repositionable external beam of radiation.

More recently, efforts have been made to provide image guidance immediately before radiation delivery or in real-time during radiation delivery. Such image-guided radiotherapy (“IGRT”) may, for example, comprise orthogonal X-ray cameras that visualize the position of tracked reference points immediately before treatment and/or in real-time. The tracked reference points may be boney landmarks, external frames and/or implanted fiducials, such as gold screws or seeds. Image guidance data may be combined with higher resolution pre-treatment MRI and/or CT data to accurately direct radiation to the target tissue.

Advantageously, real-time image guidance data may reduce or eliminate a need to immobilize the patient, since a computerized control loop may correct for intra-fractional movement of the target tissue, e.g., due to patient movement, breathing, pulsatile blood flow, etc., and may dynamically realign the radiation beam to account for such movement. Furthermore, real-time correction of radiation delivery error may allow IGRT systems to be used in the treatment of a wider variety of ailments, including those affecting moving target tissue or target tissue that is relatively distant from rigid/fixed reference points. Commercially available IGRT systems include, for example, the Novalis Tx™ (Varian Medical Systems, Inc.; Palo Alto, Calif.), TomoTherapy® (TomoTherapy Incorporated, Madison, Wis.), Synergy® (Elekta AB; Stockholm, Sweden) and the CyberKnife® (Accuray Incorporated; Sunnyvale, Calif.).

Hypertension, heart failure and chronic kidney disease represent significant and growing global health issues. Current therapies for these conditions include non-pharmacological, pharmacological and device-based approaches. Despite this variety of treatment options, the rates of control of blood pressure and the therapeutic efforts to prevent progression of heart failure and chronic kidney disease and their sequelae remain unsatisfactory. Although the reasons for this situation are manifold and include issues of non-compliance with prescribed therapy, heterogeneity in responses in terms of both efficacy and adverse event profile (e.g., side effects), significant invasiveness of device-based intervention, and others, it is evident that alternative options are required to supplement the current therapeutic treatment regimes for these conditions.

Reduction of sympathetic renal nerve activity (e.g., via denervation) can reverse these processes. Ardian, Inc., of Palo Alto, Calif., has discovered that an energy field can initiate renal neuromodulation via denervation caused by irreversible electroporation, electrofusion, apoptosis, necrosis, ablation, thermal alteration, alteration of gene expression or another suitable modality.

### SUMMARY OF THE INVENTION

The following summary is provided for the benefit of the reader only, and is not intended to limit the disclosure in any way. The present disclosure describes methods and apparatus for renal neuromodulation via stereotactic radiotherapy. Renal neuromodulation may be beneficial in the treatment of conditions or diseases associated with elevated central sympathetic drive, including hypertension, heart failure, chronic kidney disease, insulin resistance, diabetes and/or metabolic syndrome. Renal neuromodulation may be achieved by locating afferent and/or efferent renal sympathetic nerves and then utilizing stereotactic radiotherapy to expose at least some of these nerves to a radiation dose sufficient to reduce neural activity along the nerves.

A neural location element may be provided for locating certain target renal nerves, or a target region of tissue that contains target renal nerves. A stereotactic radiotherapy system may be provided for exposing the targeted renal nerves or target region of tissue to a radiation dose sufficient to reduce neural activity, with reduced or minimized radiation exposure in adjacent tissue relative to the target renal nerves or target tissue region. For the purposes of the present application, it should be understood that the terms target or targeted renal nerve(s), renal nerve target(s), target or targeted region(s) of tissue, and target or targeted tissue volume(s) may be used interchangeably to describe one or more tissue volumes containing certain afferent and/or efferent renal sympathetic nerves to be modulated.

Renal nerves may be located and targeted at the level of the ganglion and/or at postganglionic positions, as well as at preganglionic positions. Upon selection of the renal nerves (e.g., renal nerve segments) to be located and targeted, a 3-dimensional coordinate system suitable for controlled, stereotactic radiation delivery to those renal nerves may be established. Multiple reference points, preferably fixed relative to the target renal nerves, may be tracked to establish or maintain the 3-dimensional coordinate system.

The distance and direction vectors between the reference points and the target renal nerves (and/or between the reference points themselves) may be determined or specified to locate the nerves via tracking of the multiple reference points. Such vector determination may occur pre-treatment, in real-time during treatment and/or via statistical probability. Reference point tracking during or immediately prior to radiation delivery may be combined with statistical data or with higher resolution pre-treatment data that specifies these fixed vectors separating the reference points and the nerves to accurately localize the position of the target renal nerves relative to the tracked reference points and to direct radiation to the target renal nerves. Preferably, reference points may be tracked in real-time to correct for intra-fractional migration of renal nerve target(s) relative to the stereotactic radiotherapy system, e.g., due to the cardiac cycle, pulsatile blood flow, respiration, patient movement, etc.

Once target renal nerves have been selected, a 3-dimensional coordinate system has been established, and the position of the target renal nerves within the coordinate system (e.g., relative to tracked reference points) has been resolved, renal neuromodulation may proceed using the stereotactic radiotherapy system, e.g., using an image-guided radiotherapy system. Characteristics of the neuromodulatory radiotherapy session preferably are planned in advance, e.g., to determine a desired radiation dose, to accurately define the targeted tissue volume containing the target renal nerves, to determine whether radiation will be delivered in multiple fractions or in a single fraction, to reduce or minimize radiation exposure in adjacent or non-target tissue, to reduce or minimize treatment time, etc.

### BRIEF DESCRIPTION OF THE DRAWINGS

FIG. 1 is a conceptual illustration of the sympathetic nervous system (SNS) and how the brain communicates with the body via the SNS.

FIG. 2 is an enlarged anatomic view of nerves innervating a left kidney to form the renal plexus surrounding the left renal artery.

FIG. 3 is a schematic view of a commercially available image guided radiotherapy system.

FIGS. 4A and 4B are schematic views illustrating stereotactic radiotherapy applied to renal nerve targets in a vicinity of a renal artery to partially or completely denervate a kidney innervated by the targeted renal nerves with minimal or no radiation damage in adjacent tissue.

FIGS. 5A to 5E are schematic views illustrating stereotactic radiotherapy applied to additional or alternative renal nerve targets to partially or completely denervate a kidney innervated by the targeted renal nerves with minimal or no radiation damage in adjacent tissue.

FIG. 6 is an anatomic view, partially in section, of the intravascular delivery through the femoral artery and into a renal artery of a catheter having a distal region with an expandable element for expanding introduced reference points into contact with a luminal surface of the renal artery.

FIGS. 7A to 7E are detail anatomic views, partially in section, illustrating multiple exemplary embodiments of the distal region of the catheter of FIG. 6 upon expansion of the introduced reference points into contact with the luminal surface of the renal artery.

FIGS. 8A and 8B are detail anatomic views, partially in section, of a renal artery illustrating the delivery and deployment of introduced reference points that are implanted within the renal artery.

FIGS. 9A and 9B are detail anatomic views, partially in section, of a renal artery illustrating, respectively, intravascular catheter-based and extravascular needle-based methods and apparatus for the delivery of introduced reference points or contrast into an extravascular space surrounding the renal artery.

FIGS. 10A to 10D are a detail isometric view and multiple cross-sectional views of a renal artery, illustrating a plurality of longitudinally- and angularly-spaced concentric extracircumferential annular segment treatment zones that have been exposed to stereotactic radiotherapy to partially or completely denervate a kidney innervated by the targeted renal nerves with minimal or no radiation damage in adjacent tissue.

FIGS. 11A and 11B are, respectively, a detail isometric view and a cross-sectional view of a renal artery, illustrating a concentric extracircumferential annular treatment zone that has been exposed to stereotactic radiotherapy to partially or completely denervate a kidney innervated by the targeted renal nerves with minimal or no radiation damage in adjacent tissue.

FIG. 12 is a detail isometric view of a renal artery, illustrating precise delivery of radiation to a localized and tracked target segment of the renal plexus to partially or completely denervate a kidney innervated by the targeted renal nerves with minimal or no radiation damage in adjacent tissue.

### DETAILED DESCRIPTION

The present disclosure describes methods and apparatus for renal neuromodulation via stereotactic radiotherapy. Renal neuromodulation may be beneficial in the treatment of conditions or diseases associated with elevated central sympathetic drive, including hypertension, heart failure, chronic kidney disease, insulin resistance, diabetes, metabolic syndrome, sleep apnea, atrial fibrillation, and/or dyspnea.

Although this disclosure is detailed and exact to enable those skilled in the art to practice the disclosed technologies, the physical embodiments herein disclosed merely exemplify the various aspects of the invention, which may be embodied in other specific structure. While preferred embodiments have been described, the details may be changed without departing from the invention, which is defined by the claims.

Reference throughout this specification to “one example,” “an example,” “one embodiment” or “an embodiment” means that a particular feature, structure, or characteristic described in connection with the example is included in at least one example of the present disclosure. Thus, the occurrences of the phrases “in one example,” “in an example,” “one embodiment” or “an embodiment” in various places throughout this specification are not necessarily all referring to the same example. Furthermore, the particular features, structures, routines, steps or characteristics may be combined in any suitable manner in one or more examples of the disclosure. The headings provided herein are for convenience only and are not intended to limit or interpret the scope or meaning of the claimed disclosure.

### I. Pertinent Anatomy and Physiology

A. The Sympathetic Nervous System

The Sympathetic Nervous System (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation can elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting and increased blood pressure. Increased sweating is also seen, due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to things as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and, to a lesser extent, noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system—that is, one that operates without the intervention of conscious thought. Some evolutionary theorists suggest that the sympathetic nervous system operated in early organisms to maintain survival, as the sympathetic nervous system is responsible for priming the body for action. One example of this priming is in the moments before waking, in which sympathetic outflow spontaneously increases in preparation for action.

1. The Sympathetic Chain

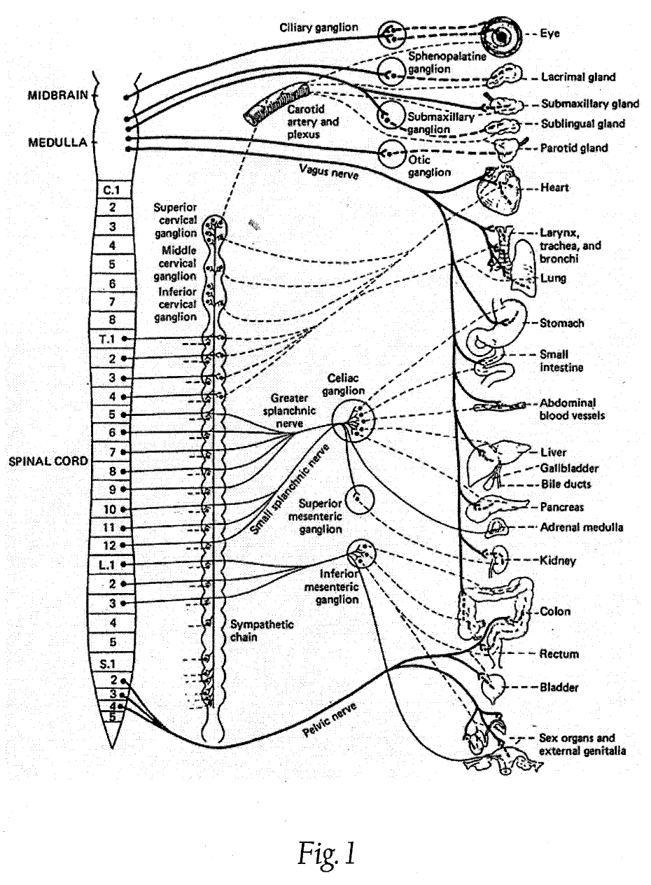

As shown in FIG. 1, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to the either the paravertebral (which lie near the vertebral column) or prevertebral (which lie near the aortic bifurcation) ganglia extending alongside the spinal column.

In order to reach the target organs and glands, the axons must travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

2. Innervation of the Kidneys

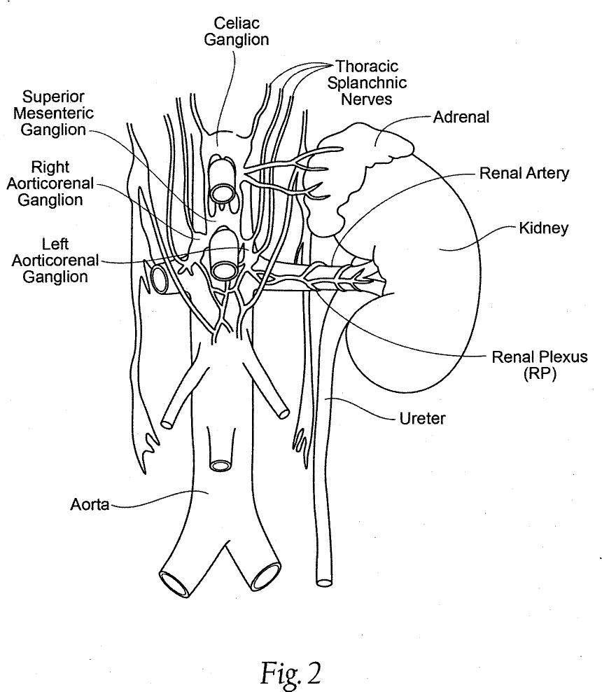

As FIG. 2 shows, the kidney is innervated by the renal plexus (RP), which is intimately associated with the renal artery. The renal plexus (RP) is an autonomic plexus that surrounds the renal artery and is embedded within or adjacent to the adventitia of the renal artery. The renal plexus extends along the renal artery until it arrives at the substance of the kidney. Fibers contributing to the renal plexus arise from the celiac ganglion, the superior mesenteric ganglion, the aorticorenal ganglion and the aortic plexus. The renal plexus (RP), also referred to as the renal nerve or nerves, is predominantly comprised of sympathetic components. There is no (or at least very minimum) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

3. Renal Sympathetic Neural Activity

Messages travel through the SNS in a bidirectional flow. Efferent messages can trigger changes in different parts of the body simultaneously. For example, the sympathetic nervous system can accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure, chronic kidney disease, insulin resistance, diabetes, metabolic syndrome, sleep apnea, atrial fibrillation, and dyspnea are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly in young hypertensive subjects, which, in concert with increased NE spillover from the heart, is consistent with the hemodynamic profile typically seen in early hypertension and characterized by an increased heart rate, cardiac output and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all cause death and death from cardiovascular disease. This is also true for patients suffering from diabetic or contrast nephropathy. There is compelling evidence that suggests that sensory afferent signals originating from the diseased kidneys are major contributors to initiate and sustain elevated central sympathetic outflow in this patient group, which facilitates the occurrence of the well-known adverse consequences of chronic sympathetic overactivity, such as hypertension, left ventricular hypertrophy, ventricular arrhythmias and sudden cardiac death.

Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na+) reabsorption and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

(ii) Renal Sensory Afferent Nerve Activity

The kidneys communicate with integral structures in the central nervous system via renal sensory afferent nerves. Several forms of “renal injury” can induce activation of sensory afferent signals. For example, renal ischemia, reduction in stroke volume or renal blood flow, or an abundance of adenosine enzyme may trigger activation of afferent neural communication. This afferent communication might be from the kidney to the brain or might be from one kidney to the other kidney (via the central nervous system). These afferent signals are centrally integrated and result in increased sympathetic outflow. This sympathetic drive is directed towards the kidneys, thereby activating the RAAS and inducing increased renin secretion, sodium retention, volume retention and vasoconstriction. Central sympathetic overactivity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of renal efferent sympathetic nerves, e.g., via denervation of tissue containing renal efferent sympathetic nerves, will reduce inappropriate renin release, salt retention, and reduction of renal blood flow, and that (ii) modulation of renal afferent sensory nerves, e.g., via denervation of tissue containing renal afferent sympathetic nerves, will reduce the systemic contribution to hypertension through its direct effect on the posterior hypothalamus, as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Neuromodulation

As provided above, renal neuromodulation, e.g., via denervation, is likely to be valuable in the treatment of several clinical conditions characterized by increased central sympathetic drive and particularly renal sympathetic activity such as hypertension, metabolic syndrome, diabetes, insulin resistance, left ventricular hypertrophy, chronic kidney disease and end stage renal disease, inappropriate fluid retention in heart failure, cardio-renal syndrome, sleep apnea, atrial fibrillation, dyspnea and sudden death. Since the reduction of afferent neural signals contributes to the systemic reduction of sympathetic tone/drive, renal neuromodulation might also be useful in treating other conditions associated with systemic sympathetic hyperactivity. Accordingly, renal neuromodulation can also benefit other organs and bodily structures innervated by sympathetic nerves, including those identified in FIG. 1. For example, a reduction in central sympathetic drive may reduce the insulin resistance that afflicts people with metabolic syndrome and Type II diabetics. Additionally, patients with osteoporosis are also sympathetically activated and might also benefit from the down-regulation of sympathetic drive that accompanies renal neuromodulation.

### II. Stereotactic Radiotherapy for Renal Neuromodulation

A. Overview

In accordance with the present application, renal neuromodulation, e.g., via denervation of tissue containing renal nerves, may be achieved by locating target renal nerves or tissue known to contain target renal nerves and then utilizing stereotactic radiotherapy to expose the target renal nerves to a radiation dose sufficient to reduce neural activity along the nerves. For the purposes of the present application, it should be understood that the terms target or targeted renal nerve(s), renal nerve target(s), target or targeted region(s) of tissue, and target or targeted tissue volume(s) may be used interchangeably to describe one or more tissue volumes containing certain afferent and/or efferent renal sympathetic nerves to be modulated.

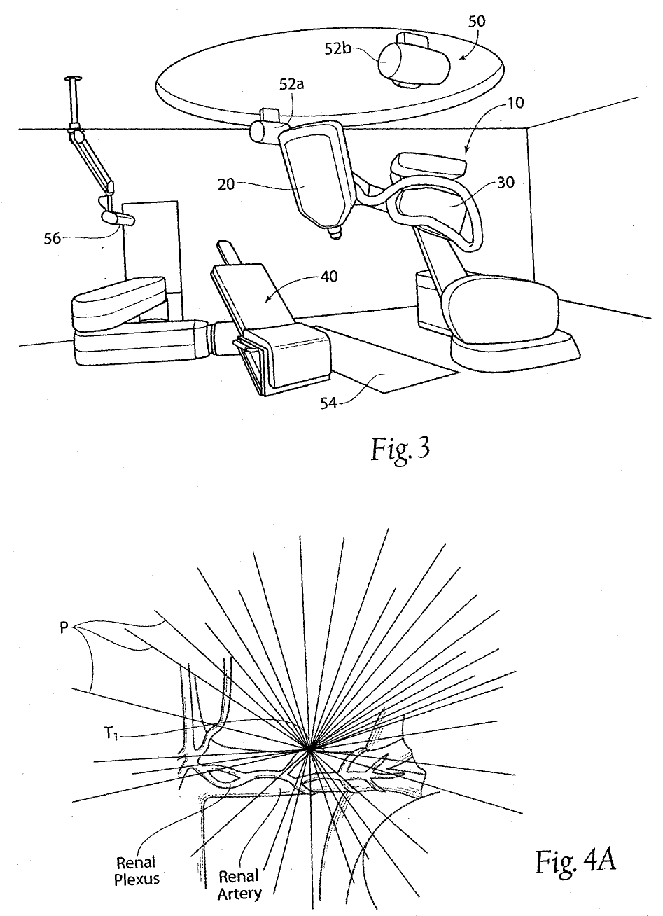

In some embodiments, a neural location element may be provided for locating target renal nerves. A stereotactic radiotherapy system (such as system 10 of FIG. 3 described below) may be provided for exposing the located target renal nerves to a radiation dose sufficient to reduce neural activity. Renal nerves may be located and targeted at the level of the ganglion and/or at postganglionic positions, as well as at preganglionic positions.

With reference to FIG. 2, ganglionic target positions may include the superior mesenteric ganglion, the aorticorenal ganglion and/or the celiac ganglion (about 40% of renal nerves extend from the celiac ganglion). Ganglionic targets may be of sufficient size or volume to achieve direct visualization of the targets via pre-treatment MRI, CT, PET or other high-resolution visualization modality. Furthermore, ganglionic targets may be relatively fixed, i.e. may not migrate significantly, relative to reference points that can be visualized in real-time and/or just prior to treatment, e.g., with an image guided radiotherapy (“IGRT”) systems. As such, a neural location element used to locate renal nerve targets may comprise a visualization modality configured to locate reference points that are substantially fixed relative to the renal nerve targets. The reference points may comprise naturally occurring anatomical reference points, for example, points along the human spine (e.g., vertebral bodies), aorta, renal arteries, kidneys, and/or the ganglia themselves, and/or may be reference points introduced by a medical practitioner. Contrast agents optionally may be delivered orally, by IV, or locally in the vicinity of the renal nerve targets (e.g., via image-guided needle injection or via catheter-based injection) to facilitate visualization of the renal nerve targets and/or the reference points.

Postganglionic renal nerves tend to extend between the ganglia and the kidneys along the renal arteries as part of the renal plexus within or adjacent the adventitia of the arterial walls. Thus, vascular anatomical landmarks may be used to locate and target (or to aid in the location and targeting of) the renal nerves. Such vascular anatomical landmarks include, but are not limited to, an intersection of a renal artery and the descending aorta; a renal artery itself, e.g., a specified outwardly directed radial distance from a luminal surface of the renal artery, adventitia of the renal artery, a medial/adventitial interface of the renal artery, distal bifurcations/branches of the renal artery, etc.; and combinations thereof.

It is believed that many renal nerves tend to reside at a transitional intersection of a renal artery and the descending aorta. As compared to more distal renal arterial segments, this intersection (also known as the renal artery ostium) may be less prone to migration relative to other anatomical structures caused by respiration, the cardiac cycle, pulsatile blood flow, patient movement, etc. Such relative immobility may aid in accurate and precise location and targeting of the renal nerves or tissue thought to contain target renal nerves.

Upon selection of the renal nerves (e.g., segments of the renal nerves) or the tissue region(s) thought to contain the renal nerves to be located and targeted, a 3-dimensional coordinate system suitable for controlled, stereotactic radiation delivery to those renal nerves may be established. Stereotactic radiotherapy systems can be configured to establish a 3-dimensional coordinate system with an isocenter that may be fixed relative to the radiotherapy system (in which case the patient may be immobilized during treatment), or the isocenter may be dynamically defined by the real-time location of targeted tissue (in which case the patient may be allowed at least limited motion during treatment).

Multiple reference points of known distance and direction vector to the targeted tissue, such as boney landmarks, fixed external frames and/or implanted fiducials (e.g., gold screws or seeds), may be tracked to locate the position of the targeted tissue relative to the tracked reference points. IGRT systems may track reference points immediately before radiation therapy and/or in real-time. Image guidance data may be combined with higher resolution pre-treatment data, such as MRI, CT and/or PET data, to accurately localize the position of the target tissue relative to the tracked reference points and to direct radiation to the targeted tissue.

The volumes of renal nerve targets and targets in accordance with the methods and systems disclosed herein are expected to be significantly smaller than any target tissue volumes previously treated using stereotactic radiotherapy; for example, while target tissue volumes in previous stereotactic radiotherapy procedures may typically be on the order of cubic centimeters, a renal nerve target of the present application may have a volume on the order of cubic millimeters. In one embodiment, each renal nerve target may comprise a tissue volume less than about 50 mm³. Additionally, some renal nerve targets may migrate significantly relative to boney structures, external frames and/or fiducials implanted in boney structures or in soft tissue, potentially complicating accurate and precise targeting of the nerves relative to such reference points. For at least these reasons, the isocenter of the 3-dimensional coordinate system preferably is dynamically defined relative to targeted renal nerves, or relative to tracked reference points that are substantially fixed relative to the targeted renal nerves. Optionally, the isocenter may move or migrate relative to the stereotactic radiotherapy system, which may correct or compensate for such relative migration in real-time.

While boney landmarks, fixed frames and/or fiducials may be tracked to establish or maintain the 3-dimensional coordinate system, as well as to locate or target the renal nerves, additional and/or alternative reference points also may be tracked in accordance with the present application. Generally, approximately 3 reference points of known vector to the target renal nerves (and/or of known vector to one another) are tracked to enable localization of the target renal nerves. The 3 reference points preferably are offset by at least 15 degrees from one another.

Tracked reference points may comprise naturally-occurring anatomical markers, such as points along the human spine (e.g., vertebral bodies), aorta, renal arteries, renal artery branching, renal vein, kidneys, and/or the renal nerves themselves. Additionally or alternatively, the tracked reference points may comprise internally and/or externally introduced reference points, such as fixed external frames, external markers attached to the patient's skin, implanted radiopaque elements such as fiducials (screws or seeds, e.g., gold), implanted magnetic elements or transponders, catheter-based or catheter-delivered reference points, needle-based or needle-delivered reference points, and/or combinations thereof. Internally introduced reference points may be positioned relative to target renal nerves using intravascular (e.g., catheter-based), extravascular (e.g., minimally invasive surgical or needle-based) or intra-to-extravascular (e.g., catheter-based) techniques. Contrast agents may be delivered orally, by IV, or locally in the vicinity of the renal nerve targets or tracked reference points (e.g., via needle-based injection or via catheter-based injection) to facilitate visualization of the renal nerve targets and/or the tracked reference points. Furthermore, internally introduced reference points may be permanently positioned within the patient or may be positioned in the patient temporarily and then removed after treatment.

When using tracked reference points to establish the 3-dimensional coordinate system suitable for controlled, stereotactic radiation delivery to target renal nerves, the target renal nerves or tissue thought to contain the target renal nerves must be located or localized relative to the tracked reference points. The tracked reference points preferably are fixed relative to the target renal nerves/tissue, and localization of the target renal nerves/tissue relative to the tracked reference points may comprise specifying or determining the length and direction vector separating one or more of the tracked reference points from the target renal nerves/tissue and/or from one another. Localization of the target renal nerves/tissue relative to the tracked reference points may occur prior to treatment, in real-time during the treatment and/or using a statistical approach to probabilistically estimate the location of the target nerves relative to the tracked reference points that establish or maintain the 3-dimensional coordinate system. When using pre-treatment localization, high-resolution MRI, CT, PET or other data, etc., may be used to determine the relative positions of the renal nerve target(s) and the reference points, which then may be tracked in real-time during the treatment.

When using a statistical approach, the renal nerves may, for example, be statistically located relative to an interior luminal surface or interior wall of the renal artery. Renal nerves generally are located between about 0 mm and about 3 mm radially distant or outward from the luminal surface, and in some patients between about 0.5 mm and about 2.5 mm radially distant or outward from the luminal surface. Thus, tracked reference points may comprise multiple points in contact with the luminal surface of the renal artery, or of known vector from the luminal surface of the renal artery. The target tissue volume for radiation therapy may comprise an extracircumferential tissue volume, such as a point or small sphere, an annulus or one or more annular segments, located between about 0 mm and about 3 mm radially distant or outward from the known position of the luminal surface. In one embodiment, the extracircumferential tissue volume is located between about 0.5 mm and about 2.5 mm radially distant or outward from the known position of the luminal surface.

Preferably, reference points may be tracked in real-time to correct for intra-fractional migration of the reference points relative to the stereotactic radiation system (e.g., due to the cardiac cycle, pulsatile blood flow, respiration, patient movement, etc.), and thereby to correct for intra-fractional migration of the renal nerve target(s) that are of fixed/known vector to the tracked reference points. Localization of the renal nerve targets and/or tracking of the reference points may utilize, for example, internal or external visual or other markers, imaging-based techniques, orthogonal X-ray cameras, fluoroscopy, MRI or functional MRI, CT, PET, magnetic or transponder techniques, radiopaque markers, catheter-based markers, temporary or permanent intravascular markers, intravascular ultrasound (“IVUS”), elastography, palpography, virtual histology, guided IVUS, pullback IVUS, optical coherence tomography, magnetic markers, ultrasound-based Time-of-Flight markers, mapping of neural response, nerve stimulation, combinations thereof, or any other method or apparatus for location and/or tracking of the renal nerve targets.

Once target renal nerves have been selected, a 3-dimensional coordinate system has been established, and the position of the target renal nerves within the coordinate system (e.g., relative to tracked reference points) has been resolved, therapy may proceed using a stereotactic radiotherapy system (e.g., using an IGRT system). FIG. 3 is a schematic view of a commercially available image guided radiotherapy system, such as the CyberKnife® system from Accuray Incorporated (Sunnyvale, Calif.). The system 10 comprises a linear accelerator (e.g., a 6MV linear accelerator) or LINAC 20 mounted on a six degree-of-freedom robotic controller 30. The LINAC 20 optionally comprises a variable aperture collimator for varying the size of the X-ray beam, as desired. The patient is situated on a patient positioning system 40, which also may comprise six degrees of freedom for faster patient setup. The imaging system 50 includes orthogonal X-ray cameras 52a and 52b that interact with flush mounted detectors 54 for real-time imaging data, as well as an optional respiratory tracking system 56 for synchronizing beam delivery to intra-fractional motion or migration of the targeted tissue volume.

A stereotactic radiation therapy session for renal neuromodulation, e.g., denervation, preferably is planned in advance, e.g., to determine a desired radiation dose, to accurately define the targeted tissue volume, to determine whether radiation will be delivered in multiple fractions or in a single fraction, to reduce or minimize radiation exposure in adjacent tissue, to reduce or minimize treatment time, etc. In one embodiment, the desired radiation dose is less than about 90 Gy. In another embodiment the desired dose is between about 60-90 Gy. In another embodiment, the desired dose is less than about 60 Gy. Preferably, the dose delivered to the target renal nerves is about the minimum dose necessary to reduce renal neural activity, e.g., to cause apoptosis and ultimately necrosis of renal sympathetic nerves, to achieve a desired therapeutic effect, such as a reduction in systolic and/or diastolic blood pressure of at least 10 mmHg.

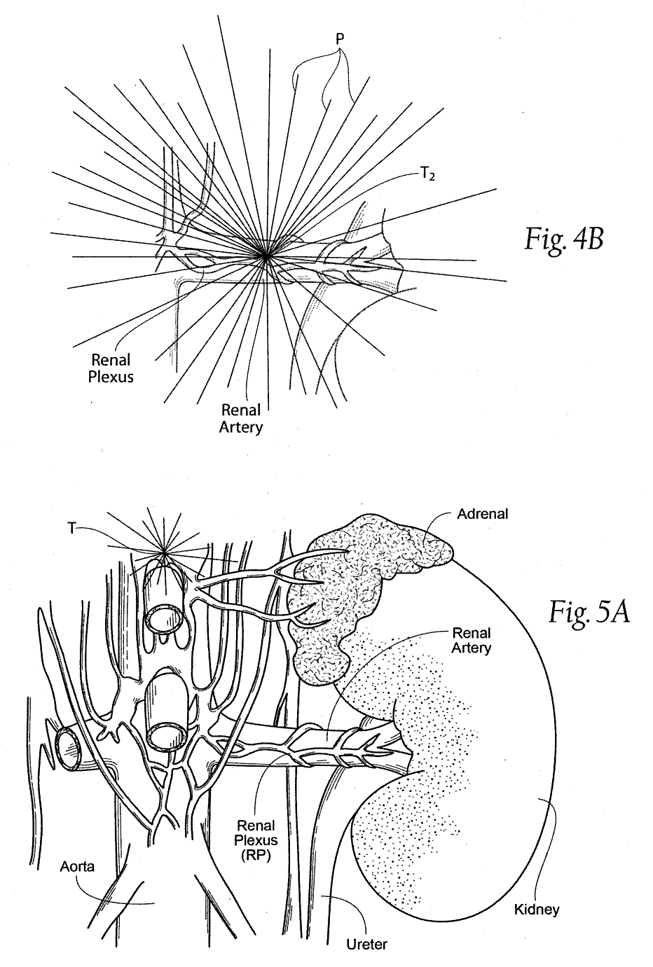

Referring now to FIGS. 4A and 4B, a stereotactic radiation therapy session is described. As seen in FIG. 4A, a renal nerve target or tissue region thought to contain target renal nerves T₁, such as a point or small volume along the renal plexus disposed between about 0 mm and 3 mm, e.g., between about 0.5 mm and about 2.5 mm, radially outward from a intraluminal surface of the renal artery, is stereotactically irradiated with a plurality of relatively low radiation dose pulses P delivered from a plurality of directions. While the figures show a plurality of radiation dose pulses P delivered in one plane, it should be understood that radiation can be delivered three dimensionally across many other planes. The cumulative radiation dose delivered to the renal nerve target T₁ is sufficient to neuromodulate target renal nerves, e.g., to reduce neural activity and/or to at least partially denervate the kidney innervated by the irradiated renal nerves. However, the stereotactic (multi-directional, low dose pulses) delivery of the target radiation dose advantageously provides a steep fall-off gradient in radiation exposure that reduces or minimizes radiation damage in adjacent and/or non-target tissue. In one embodiment, stereotactic radiation is delivered in a manner that reduces or minimizes radiation exposure in all adjacent tissue. In another embodiment, the stereotactic radiation is delivered in a manner that preferentially reduces or minimizes radiation exposure in anatomical structures deemed most critical, for example, the renal arteries themselves, the kidneys, adrenals, the aorta and/or the lymph nodes. In yet another embodiment, the stereotactic radiation beams are delivered at angles and orientations to avoid non-target tissue(s). This aspect is particularly useful in protecting blood vessels, which have luminal surfaces and epithelial cells that are more susceptible to damage via radiation. One or more additional renal nerve targets or tissue regions thought to contain target renal nerves, such as renal nerve target T₂ of FIG. 4B, optionally also may be stereotactically irradiated to reduce neural activity and/or at least partially denervate the kidney innervated by the irradiated renal nerves.

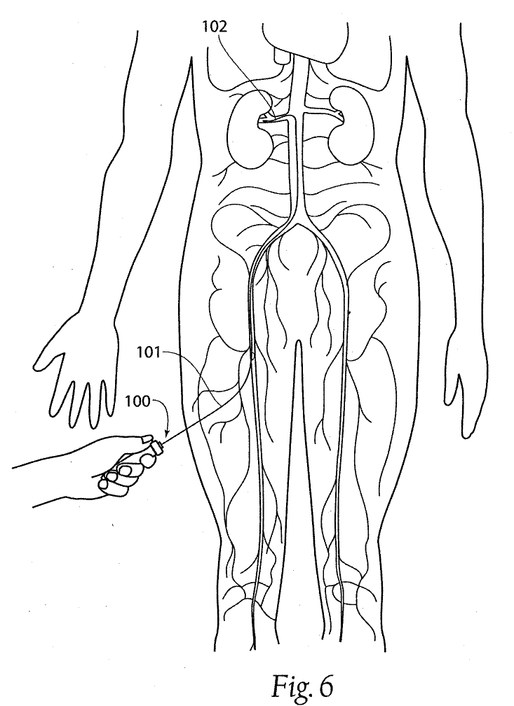

When utilizing an IGRT system, such as system 10 of FIG. 3, to conduct the stereotactic radiation therapy session of FIG. 4, pre-treatment MRI, CT, PET or other data may be utilized to establish a 3-dimensional coordinate system comprising reference points of known (preferably fixed) length and direction vector to the renal nerve target(s), as well as to define a stereotactic radiotherapy treatment protocol for achieving at least partial renal denervation. As discussed previously and hereafter, the reference points may comprise naturally occurring anatomical reference points and/or may comprise introduced reference points (see, for example, FIGS. 6-9 below). The reference points may be tracked in real-time during the stereotactic radiotherapy procedure, e.g. via imaging system 50 of IGRT system 10, to correct for migration of the reference points, and thereby to correct for migration of the renal nerve target(s) (e.g., due to the cardiac cycle, pulsatile blood flow, respiration, patient movement, etc.), relative to the radiation beam delivered from LINAC 20.

As system 10 tracks the reference points and corrects for relative migration, the system's robotic controller 30 and/or patient positioning system 40 may dynamically reorient the LINAC 20 and/or the patient to a plurality of positions, in order to align the radiation beam at a plurality of desired orientations relative to the renal nerve target. At each of these desired orientations, one or more radiation dose pulses P are delivered, such that, at the completion of the stereotactic radiotherapy treatment, the renal nerve target has been exposed to radiation dose pulses P delivered from a plurality of directions, in accordance with the pre-defined stereotactic radiotherapy treatment plan and as seen in FIG. 4. Once treatment has been initiated, IGRT system 10 optionally may autonomously or semi-autonomously proceed with the pre-defined treatment plan.

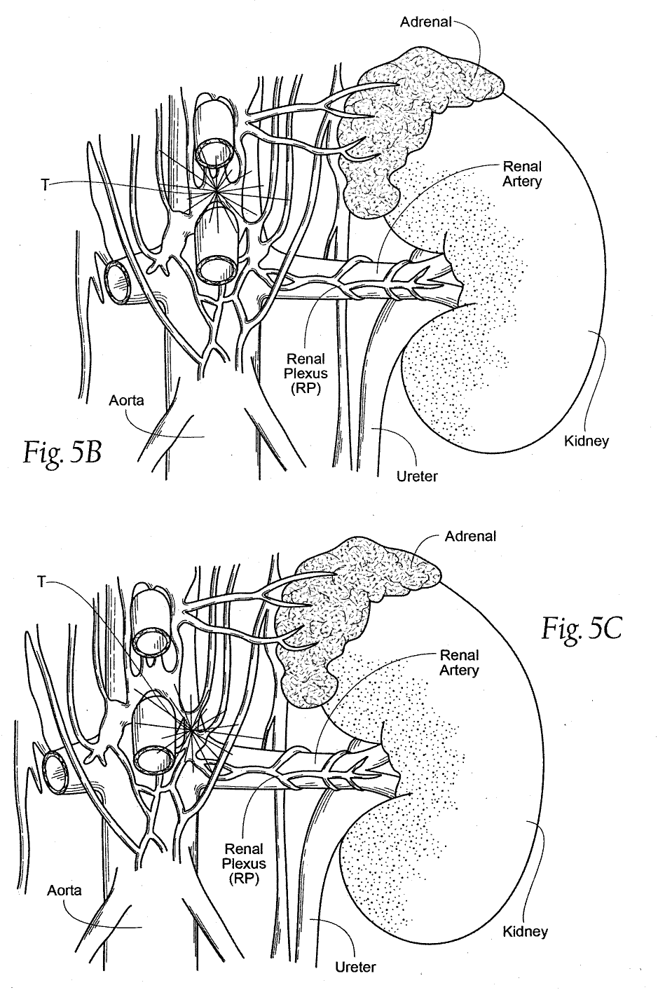

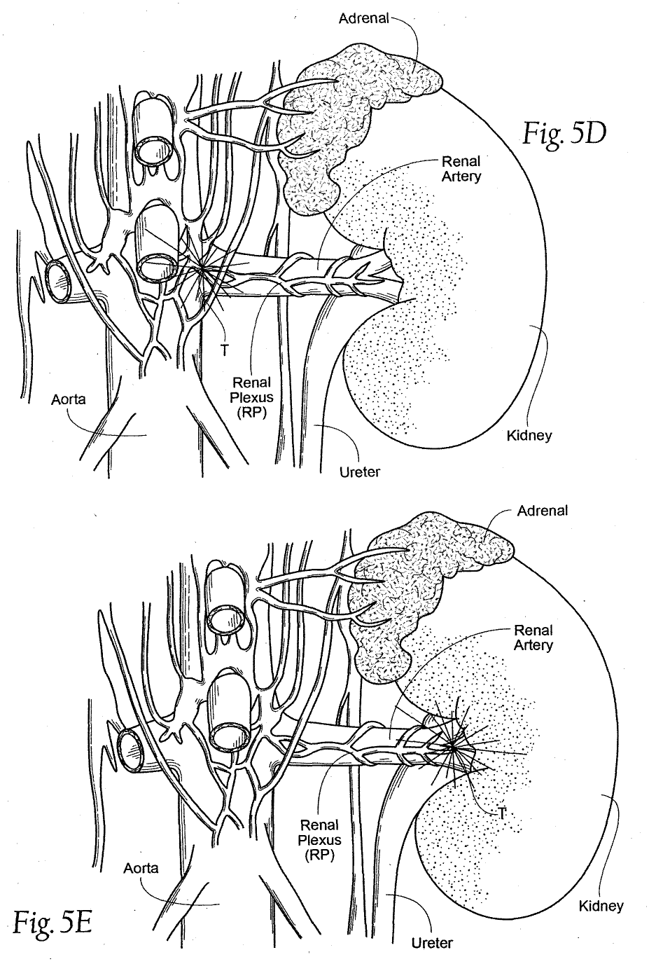

Stereotactic radiotherapy may be delivered to one or more other target sites containing renal nerves in order to achieve renal neuromodulation via at least partial renal denervation. These sites may be targeted in addition or as an alternative to the renal nerve targets positioned along the renal plexus described previously with respect to FIGS. 4A and 4B. Such additional sites include, but are not limited to, the renal nerve targets described in FIGS. 5A to 5E.

In FIG. 5A, the celiac ganglion comprises renal nerve target T that has undergone stereotactic radiotherapy with multi-directional radiation dose pulses P that reduce neural or synaptic activity in the vicinity of the renal nerve target with minimal or no radiation damage in adjacent tissue. In FIG. 5B, the superior mesenteric ganglion comprises renal nerve target T that has undergone stereotactic radiotherapy with multi-directional radiation dose pulses P that reduce neural or synaptic activity in the vicinity of the renal nerve target with minimal or no radiation damage in adjacent tissue. In FIG. 5C, the aorticorenal ganglion (illustratively, the left aorticorenal ganglion, but additionally or alternatively the right aorticorenal ganglion) comprises renal nerve target T that has undergone stereotactic radiotherapy with multi-directional radiation dose pulses P that reduce neural or synaptic activity in the vicinity of the renal nerve target with minimal or no radiation damage in adjacent tissue. In FIG. 5D, renal nerves in the vicinity of the renal artery ostium comprise renal nerve target T that has undergone stereotactic radiotherapy with multi-directional radiation dose pulses P that reduce neural or synaptic activity in the vicinity of the renal nerve target with minimal or no radiation damage in adjacent tissue. In FIG. 5E, renal nerves in the vicinity of a renal artery branching (illustratively, a branching of the left renal artery, but additionally or alternatively a branching of the right renal artery) comprise the renal nerve target T that has undergone stereotactic radiotherapy with multi-directional radiation dose pulses P that reduce neural or synaptic activity in the vicinity of the renal nerve target with minimal or no radiation damage in adjacent tissue.

B. Internally Introduced Reference Points

Tracked reference points used to conduct stereotactic radiotherapy may comprise naturally-occurring anatomical markers, such as points along the human spine (e.g., vertebral bodies), aorta, renal arteries (e.g., a specified outwardly directed radial distance from a luminal surface of a renal artery, adventitia of a renal artery, a medial/adventitial interface of a renal artery, a renal artery ostium, a renal artery bifurcation/branching, combinations thereof, etc.), kidneys, the renal nerves themselves, and/or combinations thereof. Additionally or alternatively, the tracked reference points may comprise internally and/or externally introduced reference points, such as fixed external frames, external markers attached to the patient's skin, implanted radiopaque elements such as fiducials (screws or seeds, e.g., gold), implanted magnetic elements or transponders, catheter-based or catheter-delivered reference points, needle-based or needle-delivered reference points, tracers injected into the blood stream that preferentially or specifically attach themselves to nerves, and/or combinations thereof. The reference points optionally may be tracked in real-time during the stereotactic radiotherapy procedure to correct for migration of the reference points, and thereby to correct for migration of the renal nerve target(s) (e.g., due to respiration, the cardiac cycle, pulsatile blood flow, patient movement, etc.) relative to the radiation source.

Contrast agents may be delivered orally, by IV, or locally in the vicinity of the renal nerve targets or tracked reference points (e.g., via needle-based injection or via catheter-based injection) to facilitate visualization of the renal nerve targets and/or the tracked reference points. Furthermore, substances or drugs may be delivered that work in combination with stereotactic radiotherapy to achieve desired neuromodulation. In one embodiment, such substances or drugs may be delivered in an inactive state and then put into a neuromodulatory state once delivered in a vicinity of a renal nerve target exposed to stereotactic radiotherapy.

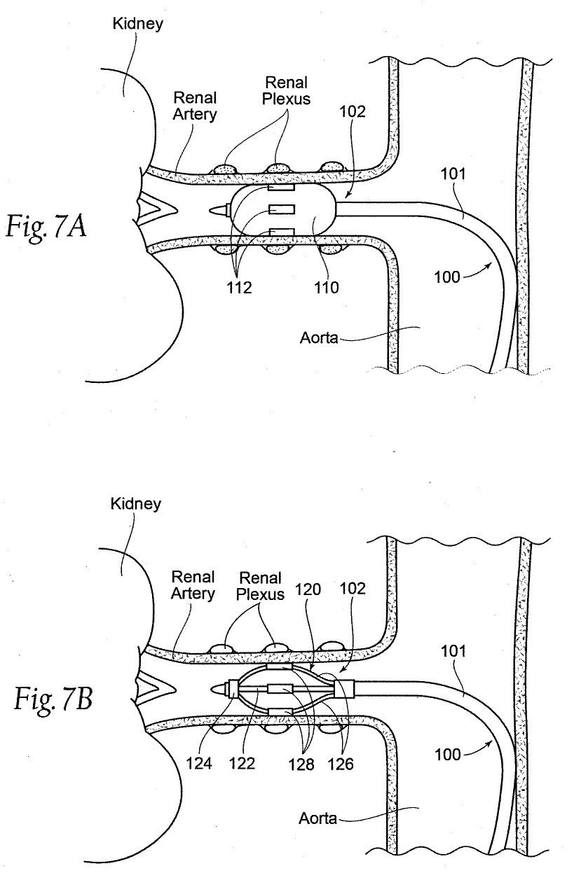

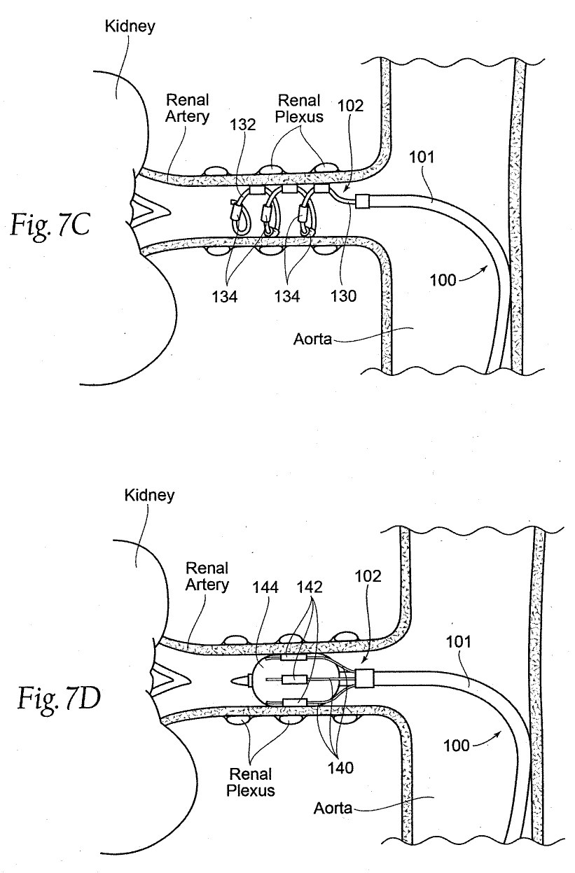

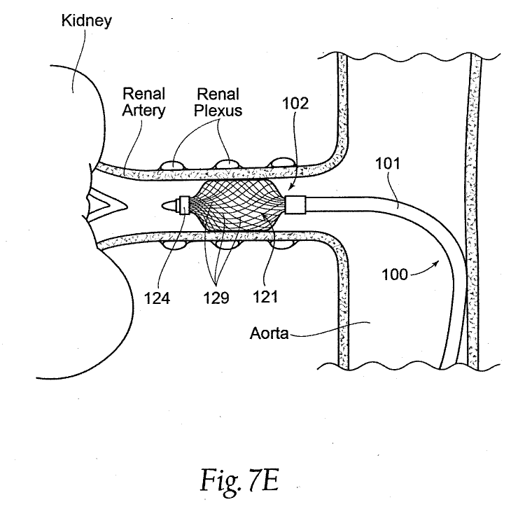

Internally introduced reference points may be positioned relative to target renal nerves using intravascular (e.g., catheter-based), extravascular (e.g., minimally invasive surgical or needle-based) or intra-to-extravascular (e.g., catheter-based) techniques. Furthermore, internally introduced reference points may be permanently positioned within the patient and/or may be positioned within the patient temporarily and then removed after treatment. Permanently positioned internal reference points may be pre-existing, such as a pre-existing renal arterial stent, may be purposely implanted for the stereotactic radiotherapy procedure, such as purposely implanted fiducial(s) or stent(s), or may be a combination of pre-existing and purposely implanted reference points. FIGS. 7-9 provide illustrative embodiments of internally introduced reference points for use during stereotactic radiotherapy to achieve at least partial renal denervation. However, it should be understood that the types and/or locations of internally introduced reference points, as well as the methods of introduction, are not limited to those shown.

With reference to FIG. 6, catheter 100 with elongated shaft 101 may be used to internally introduce reference points in the vicinity of the renal artery. The catheter comprises a distal region 102 having an expandable element for expanding introduced reference points into contact with a luminal surface of the renal artery. As seen in FIG. 6, the distal region 102 may be introduced into a patient's renal artery using well-known percutaneous techniques, e.g., may be advanced through a femoral artery access site, into the aorta, then into the right and/or left renal artery. A renal guide catheter optionally may be used to facilitate placement of the distal region 102 of the catheter 100 within the renal artery.

FIGS. 7A to 7E provide exemplary embodiments of the distal region 102 of the catheter 100 of FIG. 6, illustrating expansion of internally introduced reference points into contact with the luminal surface of the renal artery. Preferably, at least three reference points are tracked, e.g., introduced, to facilitate tracking of renal nerve target(s). The reference points may, for example, be radiopaque to facilitate X-ray visualization and are positioned temporarily within the renal artery during stereotactic radiotherapy. The renal nerve target(s) are of known length and direction vectors from the luminal surface of the renal artery contacted by the reference points (e.g., the vectors are specified, resolved, measured and/or statistically estimated), and the reference points thus may be tracked in real-time to control delivery of stereotactic radiotherapy to the renal nerve target(s).

In one embodiment, the vectors separating the renal nerve target(s) from the reference points contacting the luminal surface of the renal artery are determined by locating the relative (and relatively fixed when targeting the renal plexus) positions of the lumen of the renal artery and of the target renal nerves prior to stereotactic radiotherapy, e.g., via high-resolution MRI, CT or PET scan, or via a neural mapping or neural stimulation technique. In another embodiment, statistical probability is used to estimate the vectors separating the renal nerve target(s) and the reference points contacting the luminal wall of the renal artery. For example, a renal nerve target may be defined as a tissue volume disposed between about 0 mm and about 3 mm, e.g., about between 0.5 mm and about 2.5 mm, radially distant or outward from the luminal surface contacted by the introduced reference points.

With reference to FIG. 7A, distal region 102 of catheter 100 may comprise expandable balloon 110, such as an angioplasty or compliant balloon, having a plurality of radiopaque or other reference points 112. As shown, the reference points 112 are brought into contact with a luminal surface or internal wall of the renal artery upon expansion of the balloon. The balloon may remain inflated during stereotactic radiotherapy for renal denervation, so that the reference points 112 may be tracked to control radiation delivery, and may be deflated/collapsed and removed upon completion of the radiotherapy procedure. Subject to the length of time required to conduct the stereotactic radiotherapy, the balloon may be deflated and re-inflated once or more during the radiotherapy procedure to temporarily re-establish renal blood flow.

In FIG. 7B, the distal region 102 of catheter 100 comprises an expandable cage 120 having an elongated member 122 that is coupled to distal cap 124 and that extends proximally through a lumen of catheter 100, such that the medical practitioner may advance and retract member 122 independently of the elongated shaft 101 of the catheter 100. Cage 120 further comprises a plurality of deformable members 126 having radiopaque or other reference points 128. The deformable members 126 are distally coupled to distal cap 124 and are proximally coupled to a distal end of the shaft 101 of catheter 100. The medical practitioner may distally translate elongated member 122 relative to the elongated shaft 101 of the catheter 100 to collapse cage 120 into a low-profile delivery and retrieval configuration wherein the deformable members lie substantially flat against the elongated member 122 (not shown). When positioned with a renal artery, the elongated member 122 may be retracted proximally relative to the elongated shaft 101 of catheter 100 to cause the deformable members 126 to buckle and expand, thereby bringing reference points 128 into contact with the luminal surface or interior wall of the renal artery. The cage may remain expanded during stereotactic radiotherapy for renal denervation, and the positions of the reference points 128 may be tracked to dynamically control such therapy. Upon completion of the stereotactic radiotherapy procedure, the cage may be collapsed, and the catheter may be removed from the patient.

In one embodiment, the expanded configuration of cage 120 may be configured to move with the renal artery independently of the elongated shaft 101 of the catheter 120. In another embodiment, the cage 120 may be configured to detach temporarily or permanently from the distal end of the elongated shaft of the catheter, and optionally may be configured for future retrieval after such detachment, e.g., upon completion of stereotactic radiotherapy for renal neuromodulation. When detachable, the cage 120 optionally may be placed within the renal artery prior to the stereotactic radiotherapy procedure.

In FIG. 7C, the distal region 102 of catheter 100 comprises elongated member 130 having shaped distal segment 132. The elongated member 130 extends proximally through a lumen of catheter 100, such that the medical practitioner may advance and retract the member 130 independently of the elongated shaft 101 of the catheter 100. The shaped distal segment 132 of the elongated member 130 is configured to straighten into a reduced profile delivery configuration when disposed within the lumen of catheter 100 for delivery and retrieval of the distal region 102 of the catheter 100 within the patient's renal artery (not shown). When the elongated member 130 is advanced distally relative to the shaft 101 of the catheter 100 (or when the shaft 101 is retracted proximally relative to the elongated member 130), the shaped distal segment 132 expands into an expanded coiled, spiral, or helical shape, and a plurality of radiopaque or other reference points 134 coupled to the shaped distal segment are brought into contact with the luminal surface of the artery for tracking during stereotactic radiotherapy renal denervation. Upon completion of the procedure, the shaped distal segment may be repositioned within the lumen of the catheter 100 and removed from the patient. As with cage 120, the shaped distal segment 132 optionally may be configured for temporary or permanent separation or detachment from catheter 100 and/or from elongated member 130.

In FIG. 7D, the distal region 102 of catheter 100 comprises a plurality of elastically deformable members 140 concentrically positioned over an expandable balloon 144. The deformable members comprise a plurality of radiopaque or other reference points 142 that may be reversibly expanded into contact with the luminal surface of the renal artery via reversible expansion of the balloon for tracking during stereotactic radiotherapy for renal denervation. Upon completion of the procedure, the balloon 144 and the deformable members 140 may be collapsed for retrieval. As will be apparent to those of skill in the art, deformable members 140 alternatively may comprise self-expanding deformable members, in which case expandable balloon 144 may not be necessary and the deformable members 140 may be advanced through the lumen of catheter 100. The deformable members may be positioned within the catheter lumen during delivery and retrieval of the catheter and may be advanced distally of the catheter to facilitate self-expansion of the deformable members that brings the reference points 142 into contact with the luminal surface of the renal artery during stereotactic radiotherapy.

In FIG. 7E, the distal region 102 of catheter 100 comprises another embodiment of the expandable cage 120 of FIG. 7B. In FIG. 7E, the expandable cage comprises an expandable wire mesh or braided basket 121 with a plurality of elastically deformable wire elements that are distally coupled to the distal cap 124 of the elongated member 122, and that are proximally coupled to the distal end of the shaft 101 of catheter 100. As with cage 120 of FIG. 7B, the medical practitioner may distally translate elongated member 122 relative to the elongated shaft 101 of the catheter 100 to collapse mesh 121 into a low-profile delivery and retrieval configuration wherein the elastically deformable wire elements that form the mesh lie substantially flat against the elongated member 122 (not shown). When positioned with a renal artery, the elongated member 122 may be retracted proximally relative to the elongated shaft 101 of catheter 100 to cause the wire elements of mesh 121 to buckle and expand, thereby bringing radiopaque or other reference points 129 into contact with the luminal surface or interior wall of the renal artery. The mesh 121 may remain expanded during stereotactic radiotherapy for renal denervation, and the positions of the reference points 129 may be tracked to dynamically control such therapy. Upon completion of the stereotactic radiotherapy procedure, the mesh may be collapsed, and the catheter may be removed from the patient.

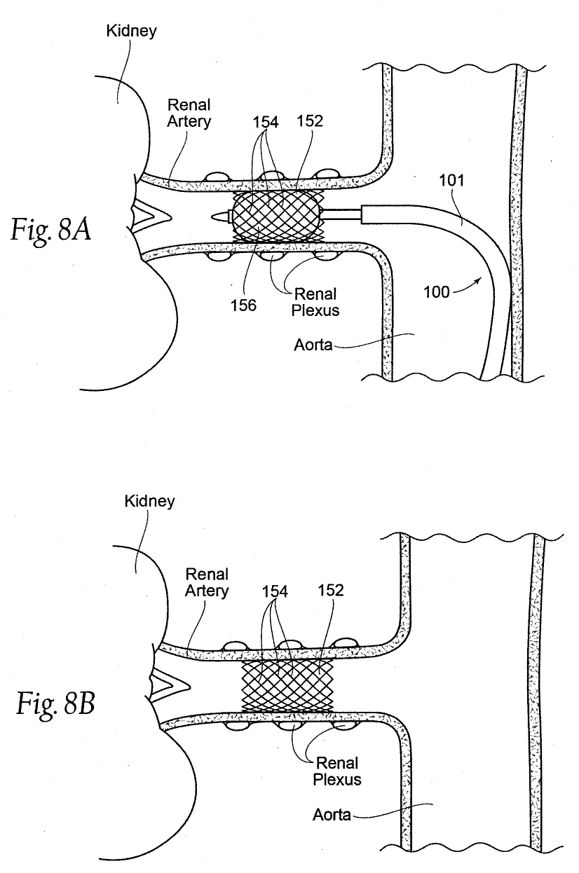

In the embodiments of FIGS. 7A-7E, the internally introduced reference points are positioned within the renal artery temporarily via a catheter-based approach. In the embodiment of FIGS. 8A and 8B the introduced reference points are temporarily or permanently implanted within the renal artery. As seen in FIG. 8A, the distal region 102 of catheter 100 comprises a radiopaque balloon-expandable stent 152 having a plurality of radiopaque or other reference points 154. Stent 152 initially is positioned over expandable balloon 156 in a low profile delivery configuration (not shown). When positioned within the renal artery, the balloon 156 is inflated to expand stent 152 and reference points 154 into contact with the luminal surface of the renal artery. Prior to stereotactic radiotherapy, the balloon 156 is deflated, and catheter 100 is removed from the patient. As seen in FIG. 8B, stent 152 remains and provides implanted introduced reference points 154 for controlling stereotactic radiotherapy for renal denervation.

As an alternative to a balloon-expandable stent, stent 152 may be comprised of a Nickel-Titanium alloy (Nitinol), which allows the stent to self-expand within the renal artery. Additionally or alternatively, stent 152 may comprise a bioresorbable material, such as polyethylene glycol. Furthermore, stent 152 optionally may be configured for retrieval and removal from the patient after completion of stereotactic radiotherapy.

Stent 152 of FIG. 8, or any of the distal regions 102 of catheter 100 shown in FIG. 7, optionally may be impregnated with contrast agent (e.g., barium) to facilitate visualization in vivo. Furthermore, the distal regions 102 of catheter 100 shown in FIG. 7 and/or the expandable stent 152 of FIG. 8 optionally may comprise radiation shielding to partially or completely shield all or part of the renal artery from radiation delivered to the target renal nerves. In one embodiment, the radiation shielding comprises a lead surface coating.

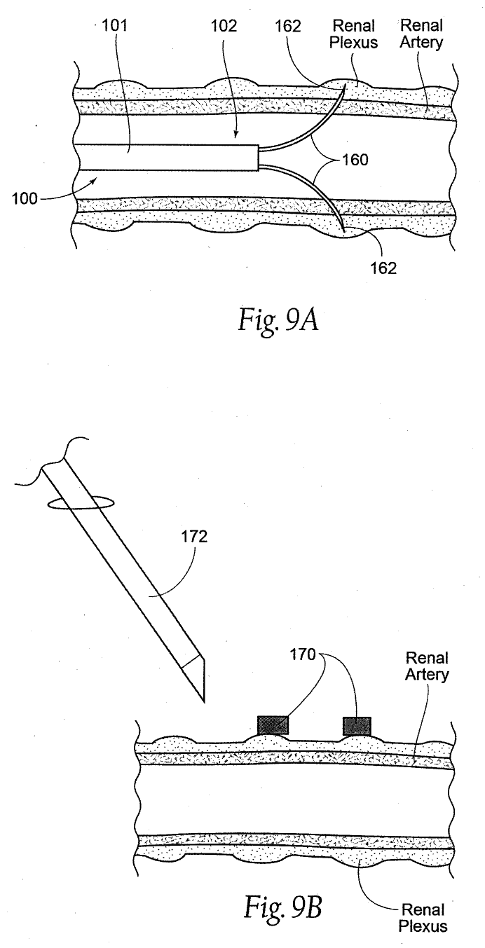

FIGS. 7 and 8 illustrate catheter-based methods and apparatus for internally introduced reference points that are temporarily positioned or permanently implanted within the patient. FIGS. 9A and 9B illustrate both catheter-based and needle-based methods for internally introducing reference points or contrast agents into an extravascular space surrounding the renal artery. In FIG. 9A, distal region 102 of catheter 100 comprises at least one needle or a plurality of shaped needles 160 that may be advanced within the lumen of the catheter and that may puncture the luminal wall of the artery and extend intra-to-extravascularly into the extravascular space surrounding the renal artery, e.g., into the adventitia of the artery. Alternatively, distal region 102 of catheter 100 may comprise a single needle configured to be positioned in the extravascular space surrounding the renal artery via an intra-to-extravascular approach. Optionally, the tips 162 of the needles 160 may comprise radiopaque or other reference points that may be tracked during stereotactic radiotherapy. Alternatively, fiducial reference points (see FIG. 9B), such as gold seeds, may be delivered through the needle tips 162 and implanted into the extravascular space for tracking during stereotactic radiotherapy for renal neuromodulation, e.g., denervation. In FIG. 9B, fiducial reference points 170 are delivered and implanted into the extravascular space under image guidance through needle 172 without puncturing the wall of the renal artery. The reference points 170 may be permanent or bioresorbable implants or, alternatively, may be configured for explantation via minimally invasive retrieval techniques. For example, reference points 170 may be implanted with fine wires that are routed to the surface of the patient's skin. Once the stereotactic radiotherapy procedure is complete, the wires may be retracted to remove the implanted reference points. The fiducial reference points 170 may be tracked during stereotactic radiotherapy for renal denervation. Contrast additionally or alternatively may be delivered into the extravascular space by shaped needles 160 of FIG. 9A or by needle 172 of FIG. 9B.

Furthermore, introduced reference points such as those described herein (FIGS. 7 to 9) may be used to provide or create reference points for use in extracorporeal procedures utilizing alternative energy modalities such as ultrasound, high intensity focused ultrasound, or lithotripsy. Medical equipment (e.g. an ultrasound generator) external to the patient may deliver energy focused on a region of tissue with a location relative to the reference point.

Introduced reference points may also be designed to interact with an extracorporeal energy source to create a neuromodulatory effect such as thermal ablation. For example, the introduced reference point may have a structure that is ferromagnetic such that external application of an alternating magnetic field in the vicinity of the introduced reference point in a patient's body will cause the ferromagnetic component to vibrate and produce heat. When the introduced reference point is next to or in close proximity to a target sympathetic nerve, the heat produced may conduct to the nerve and thermally ablate it.

Internally introduced reference points may also be useful for other treatment approaches and modalities that aim to neuromodulate, e.g. denervate, renal nerves. For example, an internally introduced reference point may facilitate introduction, positioning and placement of an extravascular treatment device. Extravascular treatment devices may comprise devices that approach renal nerves external to a patient's vasculature (e.g. percutaneous, laparoscopic and transgastric approaches) that deliver neuromodulatory energy such as radiofrequency, thermal energy, electrical stimulation or cryogenic energy. An internally introduced reference point may be positioned proximate to target renal nerves. For example, an intravascular catheter may place an expandable radiopaque basket in a renal artery (as depicted in FIG. 8A). Using an imaging modality, such as fluoroscopy, a physician may advance an extravascular treatment device to a target location relative to the reference point. In the example of an expandable radiopaque basket placed in a renal artery, a target location may be about 1 to 4 mm from the outer diameter of the basket which may represent the adventitia of a renal artery where renal nerves may reside.

Extravascular treatment devices used with internally introduced reference points may have additional features that increase safety, efficacy or ease of use. For example, an extravascular device may be a percutaneous probe that is inserted through a patient's skin and passed through tissue to a target tissue region. Such a probe may have a blunt or rounded tip that may pass through tissue such as muscle and adipose tissue but does not easily puncture or cut blood vessels or nerves. The probe may further comprise a steerable feature such as a pre-formed bend near the distal end that may allow the probe to be guided as it is advanced through tissue and rotated. A pre-formed bend or curve near the distal end of a probe may also allow an energy delivery portion of the probe to be placed around a portion of a renal artery. Alternatively, an extravascular treatment device may have a deflectable portion that is actuated by the physician using the device to facilitate introduction of the device through tissue to the target tissue region and/or to place the device in an appropriate configuration in the target tissue region. An extravascular treatment device may comprise an electrode proximate to the distal end used to measure impedance of the tissue between the electrode and a return electrode on the internally introduced reference point or a dispersive electrode placed on the patient's skin. Measured tissue impedance may be used to indicate the type of tissue that the electrode is in. Impedance of tissue between the electrode and the internally introduced reference point may indicate relative proximity between the two electrodes.

Furthermore, when used with an extravascular treatment device, an intravascular catheter used to place an internally introduced reference point may have additional features to improve safety of the procedure. For example, if the extravascular treatment device delivers thermal treatment energy to ablate renal nerves and the target tissue region is near the luminal surface of a blood vessel such as the renal artery, an intravascular catheter may comprise an internally introduced reference point and a thermal protective device to reduce the risk of the thermal treatment energy causing injury to the non-target tissue such as the epithelium and media of the renal artery. If the thermal treatment energy is increased temperature (e.g. radiofrequency, resistive heat, ultrasound, microwave) the thermal protective device may cool the inner layers of the blood vessel to maintain a non-injurious temperature; if the thermal treatment energy is decreased temperature (e.g. cryogenic ablation) the thermal protective device may warm the inner layers of the blood vessel to maintain a non-injurious temperature. The extravascular treatment device and/or the thermal protective device may have a temperature sensor to indicate tissue temperature. Furthermore, temperature data may be used to control energy delivery and/or thermal protection. When both the extravascular treatment device and the thermal protective device comprise temperature sensors the tissue temperature measured at each location may be used to predict a thermal gradient. A thermal protective device that cools tissue may be a balloon with circulating coolant such as chilled saline. A thermal protective device that warms may be a balloon with circulating warm fluid or a resistive heating element.

C. Treatment Delivery

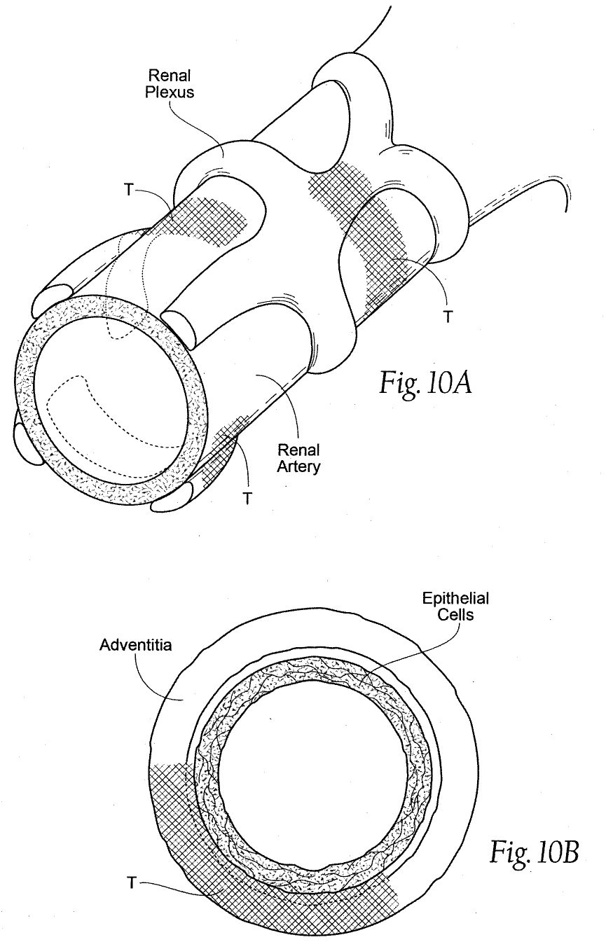

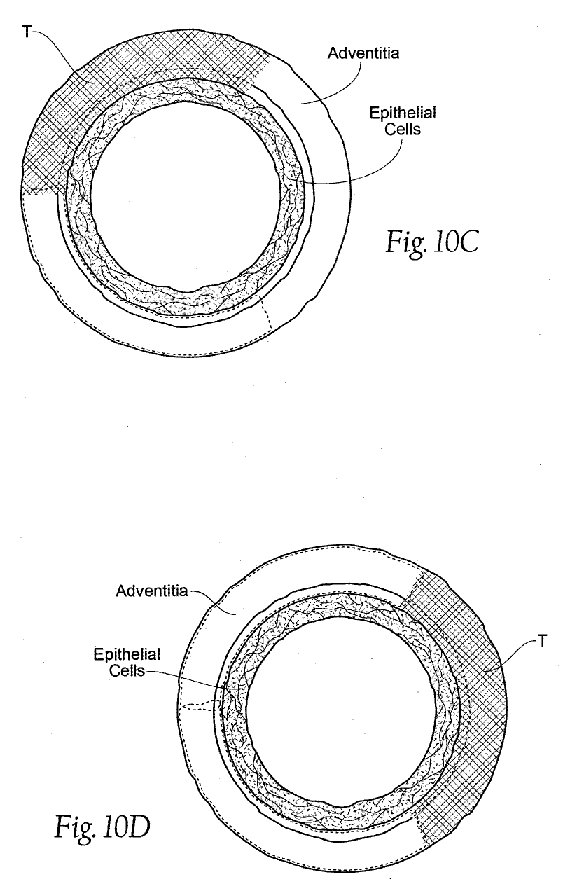

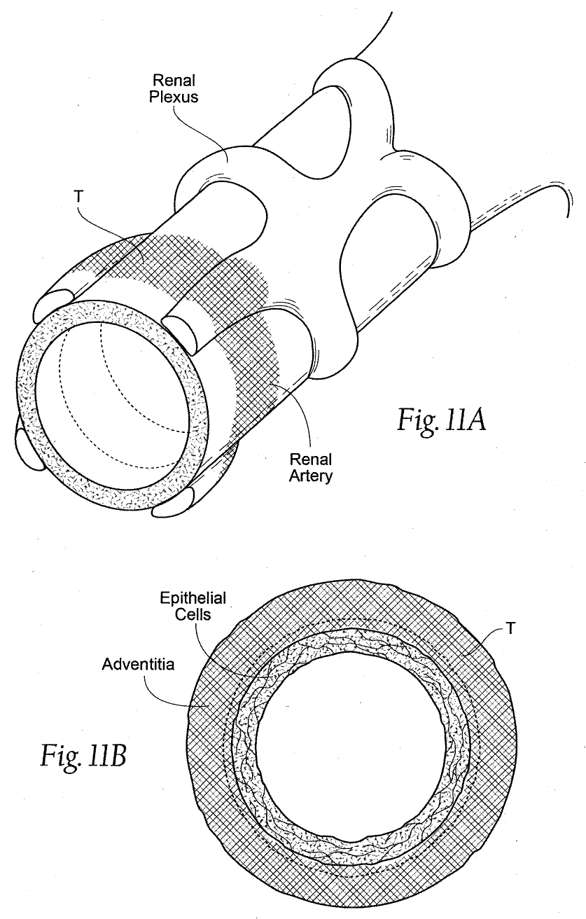

As discussed previously, when the position of the renal artery intraluminal surface is known, e.g., via tracking of internally introduced reference points as in FIGS. 7-9, and when the renal nerve target(s) are along the renal plexus, statistical probability may be used to estimate the position(s) of the renal nerve targets relative to the intraluminal surface of the renal artery. For example, a renal nerve target may be defined as a tissue volume disposed between about 0 mm and about 3 mm, e.g., between about 0.5 mm and about 2.5 mm, radially distant or outward from the luminal surface of the renal artery. As seen in FIG. 10A, a plurality of longitudinally- and angularly-spaced concentric extracircumferential annular segment target tissue volumes or treatment zones T may be defined as renal nerve targets in this way, and may be exposed to stereotactic radiotherapy to partially or completely denervate a kidney innervated by the targeted renal nerves passing through the treatment zones.

As seen in the cross-sections of FIGS. 10B to 10D, stereotactic radiotherapy delivered to the annular segment treatment zones T may kill tissue and renal nerves disposed therein within or adjacent the adventitia of the renal artery, while causing minimal or no radiation damage in adjacent vascular tissue, including sensitive epithelial cells of the renal arterial wall. Furthermore, the annular segment treatment zones T optionally may be defined as in FIG. 10, such that superimposition of the multiple angularly offset and longitudinally spaced annular segment treatment zones creates a fully annular concentric extracircumferential treatment zone. A fully annular concentric treatment zone may increase a probability of kidney denervation, as compared to one or more concentric treatment zones that collectively encompass only a partial annular segment, while longitudinal spacing of multiple annular segments without formation of a fully annular segment at any one longitudinal position along the renal artery may reduce a risk of significant injury to annularly-oriented smooth muscle cells in the arterial wall.

As seen in FIG. 11A, a concentric extracircumferential treatment zone T additionally or alternatively may be formed that encompasses a complete annulus within the adventitia of the renal artery. The treatment zone T is exposed to stereotactic radiotherapy to partially or completely denervate a kidney innervated by the renal plexus that passes through the treatment zone T. As seen in FIG. 11B, a steep fall-off gradient in the radiation dose delivered to the treatment zone T provides minimal or no radiation damage in adjacent vascular tissue, including sensitive epithelial cells of the renal arterial wall.

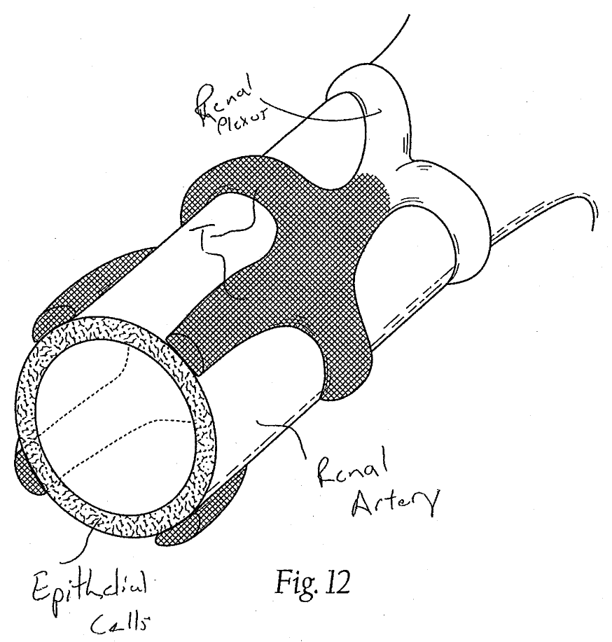

In addition to statistical approaches for defining renal nerve target(s), when the position of the renal nerves relative to tracked reference points (e.g., relative to the luminal surface of the renal artery) is known with fidelity (e.g., via pre-treatment high-resolution imaging and/or via neural mapping techniques), the renal nerve target(s) may be defined precisely. For example, as seen in FIG. 12, renal nerve target T comprises a treatment zone that conforms to the complex geometry of a segment of the renal plexus. The renal plexus is accurately and precisely exposed to stereotactic radiotherapy to partially or completely denervate a kidney innervated by the renal plexus with minimal or no radiation damage in adjacent vascular tissue, including sensitive epithelial cells of the renal arterial wall.

As discussed previously, stereotactic radiotherapy is delivered to target renal nerves in a manner that avoids excessive radiation exposure in non-target or adjacent tissue. The stereotactic radiotherapy system preferably comprises software including a control algorithm or loop, as well as a computer controller that implements instructions from the software that may be used for planning and executing the stereotactic radiotherapy procedure to achieve desired renal neuromodulation, while avoiding excessive radiation exposure in non-target or adjacent tissue. Preferably, the stereotactic radiotherapy system executes the software instructions autonomously or semi-autonomously after initiation of a stereotactic radiotherapy procedure to control and direct radiation delivery during the procedure. Preferably, the software instructions direct the stereotactic radiotherapy system to correct for intra-fractional movement of renal nerve target(s).

D. Treatment Diagnostics

Accurate and precise targeting of renal nerves is important in both ensuring stereotactic radiotherapy treatment efficacy, as well as in reducing or minimizing damage induced in adjacent, non-target tissue. Targeting may be especially important when working at the level of the spine, as any unintended nerve destruction may result in significant adverse consequences or side effects. Furthermore, when targeting ganglia, it may not be necessary to irradiate all ganglia associated with renal function.

One or more diagnostic tests may be applied to ganglionic or post-ganglionic nerve target(s) to confirm a desired physiologic effect prior to stereotactic irradiation of the target(s). Such diagnostic tests may include, but are not limited to, neural stimulation, injection of chilled saline or other cooling of the neural target, combinations thereof, etc. Various neural targets, e.g., ganglia, optionally may be tested to determine which of such neural targets would be expected to provide the greatest desired therapeutic response upon stereotactic radiotherapy. The stereotactic radiotherapy treatment protocol may be adjusted or modified subject to diagnostic test results, such that only neural target(s) expected to provide the desired therapeutic response after stereotactic radiotherapy are irradiated. Thus, diagnostic tests may limit or reduce the volume of tissue irradiated and/or the total radiation dose provided to the patient.

In one embodiment, the diagnostic test may comprise a needle electrode used to stimulate renal nerve target(s). In one embodiment, the diagnostic test may comprise a needle or needle electrode used in combination with infused temporary analgesics, such as lidocaine. In one embodiment, the diagnostic test may comprise a needle or needle electrode used in combination with cooling (e.g., infused cold saline or other fluid, cryotherapy, thermoelectric cooler elements and/or other elements for reversibly or permanently reducing the temperature of neural targets). In one embodiment, the diagnostic test may comprise a needle or needle electrode used in combination with heating (e.g., infused hot saline or other fluid, radiofrequency heating or ablation and/or other elements for reversibly or permanently reducing the temperature of neural targets). In some embodiments, the needle may comprise a lumen for delivering a fine wire electrode, infusing fluids or drugs, etc. In some embodiments, the needle itself may comprise or be an electrode (e.g., the needle may not comprise a lumen).

The above described diagnostic components and/or other sensors may be included on or associated with an internally introduced reference point such as those shown in FIGS. 6 to 9. For example, expandable cage 120 in FIG. 7B may carry one or more sensors to acquire physiologic data associated with the treatment, target tissue and/or non-target tissue. This data may facilitate positioning of the extravascular or extracorporeal treatment device as well as treatment parameters associated with use of said devices.

### III. Additional Clinical Uses of the Disclosed Apparatuses, Methods and Systems

Although much of the disclosure in the present patent application relates to renal neuromodulation by at least partially denervating a kidney of a patient to block afferent and/or efferent renal neural communication, the apparatuses, methods and systems described herein may potentially be adapted for use in treating other neuromodulation conditions, disorders or disease states. For example, the aforementioned system, or select aspects of such system, can potentially be adapted to target and deactivate neural pathways that play a role in other disease states.

Sympathetic nerves proximate to or encircling the arterial blood vessel known as the celiac trunk may pass through the celiac ganglion and follow branches of the celiac trunk to innervate the stomach, small intestine, abdominal blood vessels, liver, bile ducts, gallbladder, pancreas, adrenal glands, and kidneys. Modulating these nerves either in whole or in part via selective modulation may enable treatment of conditions including (but not limited to) diabetes, pancreatitis, obesity, hypertension, obesity related hypertension, hepatitis, hepatorenal syndrome, gastric ulcers, gastric motility disorders, irritable bowel syndrome, and autoimmune disorders such as Crohn's disease.

Sympathetic nerves proximate to or encircling the arterial blood vessel known as the inferior mesenteric artery may pass through the inferior mesenteric ganglion and follow branches of the inferior mesenteric artery to innervate the colon, rectum, bladder, sex organs, and external genitalia. Modulating these nerves either in whole or in part via selective modulation may enable treatment of conditions including (but not limited to) GI motility disorders, colitis, urinary retention, hyperactive bladder, incontinence, infertility, polycystic ovarian syndrome, premature ejaculation, erectile dysfunction, dyspareunia, and vaginismus.

### IV. Conclusion

The above detailed descriptions of embodiments of the invention are not intended to be exhaustive or to limit the invention to the precise form disclosed above. Although specific embodiments of, and examples for, the invention are described above for illustrative purposes, various equivalent modifications are possible within the scope of the invention, as those skilled in the relevant art will recognize. For example, while steps are presented in a given order, alternative embodiments may perform steps in a different order. The various embodiments described herein can also be combined to provide further embodiments.

From the foregoing, it will be appreciated that specific embodiments of the invention have been described herein for purposes of illustration, but well-known structures and functions have not been shown or described in detail to avoid unnecessarily obscuring the description of the embodiments of the invention. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the invention. Accordingly, the invention is not limited except as by the appended claims.

I/we claim:

1. A method for stereotactic radiation-based renal neuromodulation, the method comprising:
locating a region of tissue containing nerves that innervate a kidney of a patient; and
exposing the region of tissue to radiation via stereotactic radiotherapy in a manner and at a dosage sufficient to reduce neural activity in the nerves.

2. The method of claim 1 wherein locating a region of tissue comprises creating a three-dimensional coordinate system for identifying and targeting renal nerves.

3. The method of claim 2 wherein creating a three-dimensional coordinate system for identifying and targeting renal nerves comprises creating the coordinate system using at least three reference points.

4. The method of claim 3 wherein creating the coordinate system using at least three reference points comprises creating the coordinate system with at least one reference point in the vicinity of the targeted renal nerves.

5. The method of claim 1 wherein locating a region of tissue comprises locating the region of tissue using natural anatomical reference points.

6. The method of claim 5 wherein locating the region of tissue using natural anatomical reference points comprises locating the region of tissue using at least one of a renal artery, adventitia of a renal artery, renal artery ostium, renal artery branching, renal vein, kidney, aorta, vertebral bodies, or renal nerves.

7. The method of claim 1 wherein locating a region of tissue comprises locating the region of tissue using introduced reference points.

8. The method of claim 7 wherein locating the region of tissue using introduced reference points comprises positioning a fiducial marker in a vicinity of or within the tissue region.

9. The method of claim 8 wherein positioning a fiducial marker in a vicinity of or within the tissue region comprises delivering the fiducial marker via an intravascular, extravascular or intra-to-extravascular approach.

10. The method of claim 8 wherein positioning a fiducial marker in a vicinity of the tissue region comprises permanently or temporarily implanting a radiopaque fiducial marker in the vicinity of the tissue region.

11. The method of claim 8 wherein positioning a fiducial marker in a vicinity of the tissue region comprises intravascularly delivering a catheter to within a renal artery of the patient.

12. The method of claim 11 wherein intravascularly delivering a catheter to within a renal artery of the patient comprises delivering a catheter having a distal region comprising a plurality of radiopaque reference points.

13. The method of claim 1 wherein locating a region of tissue comprises visualizing the region of tissue using an imaging modality.

14. The method of claim 13 wherein visualizing the region of tissue using an imaging modality comprising visualizing the renal nerves with the region of tissue using at least one of external MRI, CT, PET, neural mapping, OCT, IVUS, elastography, virtual histology and/or intravascular MRI.

15. The method of claim 13 wherein visualizing the region of tissue using an imaging modality comprises pre-treatment visualization or real-time visualization during treatment.

16. The method of claim 1 wherein locating a region of tissue comprises employing a statistical selection approach.

17. The method of claim 16 wherein employing a statistical selection approach comprises selecting a region of tissue relative to a trackable anatomical marker based on the probability that renal nerves are located in proximity to the anatomical marker.

18. The method of claim 17 wherein selecting a region of tissue relative to a trackable anatomical marker comprises selecting a region of tissue proximate to a renal artery of the patient.

19. The method of claim 1 wherein exposing the region of tissue to radiation via stereotactic radiotherapy comprises delivering a dosage of about 60 to about 90 Gy to the region of tissue.

20. The method of claim 1 wherein exposing the region of tissue to radiation via stereotactic radiotherapy comprises delivering a plurality of radiation beams to the region of tissue from different angular positions.

21. The method of claim 20, further comprising minimizing radiation exposure to non-target tissue and/or tissue adjacent to the region of tissue.

22. The method of claim 21 wherein minimizing radiation exposure comprises delivering the radiation beams at angles and orientations to avoid non-target tissue and/or tissue adjacent to the region of tissue.

23. The method of claim 21 wherein minimizing radiation exposure comprises providing shielding on the exterior and/or interior of the patient.

24. The method of claim 1 wherein exposing the region of tissue to radiation via stereotactic radiotherapy comprises creating an extracircumferential annular treatment zone.

25. The method of claim 24 wherein creating an extracircumferential annular treatment zone comprises creating a fully extracircumferential annular treatment zone.

26. The method of claim 24 wherein creating an extracircumferential annular treatment zone comprises creating a plurality of substantially concentric extracircumferential annular treatment zone segments.

27. The method of claim 26 wherein creating a plurality of concentric extracircumferential annular treatment zone segments comprises creating a plurality of longitudinally and angularly spaced concentric extracircumferential annular treatment zone segments.

28. The method of claim 1 wherein locating a region of tissue comprises injecting contrast agent in a vicinity of the region of tissue.

29. An apparatus for renal neuromodulation, the apparatus comprising:
a neural location element for locating a tissue region comprising renal nerves; and
a stereotactic radiotherapy system configured to expose a small volume treatment region associated with the located tissue region to a radiation dose sufficient to reduce neural transduction in the small volume treatment region.

30. The apparatus of claim 29 wherein the stereotactic radiotherapy system comprises a linear accelerator and a collimator configured to deliver beam radiation to the small volume region associated with the located treatment region.

31. The apparatus of claim 29 wherein the stereotactic radiotherapy system comprises a six degree-of-freedom robotic controller configured to enable the delivery of multiple beams of radiation to the small volume treatment region from different angles and orientations.

32. The apparatus of claim 29 wherein the stereotactic radiotherapy system is configured to expose to radiation a small volume treatment region of no larger than about 50 cubic millimeters.

33. The apparatus of claim 29 wherein the neural location element comprises an imaging modality configured to facilitate visualization of renal nerves.

34. The apparatus of claim 29 wherein the neural location element further comprises an endovascular catheter configured to be positioned proximate to the tissue region.

35. The apparatus of claim 34 wherein the endovascular catheter is configured to position a fiducial reference point proximate to or within the tissue region.

36. The apparatus of claim 35 wherein the endovascular catheter comprises a distal end region that carries the fiducial reference point.

37. The apparatus of claim 36 wherein the distal end region of the catheter comprises an expandable section configured to alternate between a reduced profile delivery configuration and an expanded configuration configured to position the fiducial reference point in close proximity to the tissue region.

38. The apparatus of claim 37 wherein the expandable section in its expanded configuration is configured to place the fiducial reference point in contact with the interior wall of a renal artery.

39. The apparatus of claim 37 wherein the expandable section in its expanded configuration comprises a coiled, helical or spiral shaped section that carries the fiducial reference point.

40. The apparatus of claim 37 wherein the expandable section comprises a balloon.

41. The apparatus of claim 37 wherein the expandable section comprises a cage.

42. The apparatus of claim 41 wherein the cage comprises an expandable wire mesh or braided basket.

43. The apparatus of claim 37 wherein the expandable section comprises at least one elastically deformable member.

44. The apparatus of claim 35 wherein the catheter is configured to position an expandable stent proximate to the tissue region.

45. The apparatus of claim 44 wherein the expandable stent is impregnated with contrast agent to facilitate visualization by the imaging modality.

46. The apparatus of claim 44 wherein the expandable stent comprises a bioresorbable implant.

47. The apparatus of claim 44 wherein the expandable stent is configured to be deployed in a treatment configuration during stereotactic radiotherapy and subsequently retracted in a retrieval configuration following treatment.

48. A method of treating a patient diagnosed with a condition or disease associated with elevated central sypathetic drive, the method comprising:
selecting or identifying target renal nerves;
establishing a 3-dimensional coordinate system;
determining the position of the target renal nerves within the coordinate system; and
applying radiation to the target renal nerves with a stereotactic radiotherapy system.

49. A method of claim 48 wherein a condition or disease associated with elevated central sympathetic drive comprises at least one of hypertension, heart failure, chronic kidney disease, insulin resistance, diabetes and/or metabolic syndrome.
